(19) United States
(12) Patent Application Publication
(10) Pub. No.: US 2020/0060872 A1
(43) Pub. Date: Feb. 27, 2020

(54) Vaginal Drying Air Pump and Method, Process and System For Cooling & Drying Vagina: Reducing Excessive Moisture and Excessive Heat Within the Vaginal Area

(71) Applicants: Kellie Lanor Young, Southfield, MI (US); Kim Baneia Bradley, Detroit, MI (US)

(72) Inventors: Kellie Lanor Young, Southfield, MI (US); Kim Baneia Bradley, Detroit, MI (US)

(21) Appl. No.: 16/501,993

(22) Filed: Jul. 18, 2019

Related U.S. Application Data
(60) Provisional application No. 62/764,349, filed on Jul. 30, 2018.

(51) Int. Cl. A61F 7/12

(52) U.S. Cl. CPC A61F 7/12; A61F 2007/005

(57) ABSTRACT

The present invention and method, process & system comprises a vaginal drying air pump device/apparatus of a barrel (outer tube), inner tube, easy gripping rubber pipet/syringe air bulb and a permanent rubber seal or rubber gasket; constructed from a re-designed tampon plastic applicator with a uniquely new function and new objective, which is capable of reducing the excessive moisture and excessive heat in the vagina that causes and contributes to bacterial and vaginal yeast infections. The vaginal pump and method/process and system are designed with a manual hand-operated, squeezable syringe air bulb to produce a consistent flow of air within the vagina to dry excessive moisture and reduce excessive heat. In preferred embodiments, the various components of the vaginal pump are constructed into 2-pieces that are easily screwed together at the connecting threads by the end user. The vaginal drying air pump is reusable, easy to clean and portable.

### BACKGROUND OF THE INVENTION

The present invention relates to a portable, reusable feminine hygiene device, in particular a manual, squeezable hand-operated (non-electrical, non-electronic, non-battery operated) vaginal drying air pump. More specifically, the invention relates to a new safe device and method (other than prescription pills, suppositories and antifungal creams) that is capable of reducing and preventing excessive moisture and excessive heat, as well as lowering (or reducing) the risks of imbalanced ph conditions within the vagina that causes and/or contributes to producing bacterial and vaginal yeast infections (known as vaginitis, inflammation of the vaginal area).

Prescription pills, suppositories, antifungal creams, sticks and wands; vaginal bacterial and yeast infection treatments, have long been known and widely used by women (up to 75% in the U.S. alone) to relieve symptoms (in addition to an odorless white discharge) such as: itching in the vaginal or vulvar area, burning sensations, and pain during intercourse or urination as a result of vaginal bacterial and yeast infections. A population of half of these women will get yeast infections two or more times, within their life-time.

Over the years, the development of douches, devices, medications, and methods have all approached the problem of vaginal yeast infection by treating the symptoms after the onset of infection, but no remedy has been created to deal with the actual issue of eliminating or reducing the excessive moisture and excessive heat within the vaginal area which greatly contribute to or help to cause and/or magnify the issue of bacterial vaginal yeast infection. Using the Vaginal Drying Air Pump with the method of drying and/or removing the excessive heat and drying the excessive moisture of the vaginal area immediately offers several advantages over just using medication, suppositories or antifungal creams alone, including but not limited to the following: comfort, confidence & self-esteem, not needing to wear sanitary napkins to prevent embarrassment, enjoyable pleasurable and better intimacy or intercourse and more importantly, better and improved hygiene for eliminating and/or reducing future risks of further bacterial and vaginal yeast infections, as a result of excessive moisture and excessive heat within the vaginal area.

The terms “vagina wand” and “virgin stick” are commonly used in the industry or trade, and synonymous with other terms for devices that have been developed to reduce excessive vaginal wetness—however, these devices suffer many drawbacks that hinder the pleasure and the act of sexual intercourse and have been known to cause over-tightness which can prevent penetration of the penis and also known to cause vaginal burning due to the chemical components contained in them.

Therefore, a need exists in the field for the vaginal drying air pump device an apparatus and cooling & drying method capable of producing a better and improved vaginal environment—containing no chemicals, no electrical, no electronic, nor battery-operated components which when inserted into the vagina and hand-operated by simply squeezing the syringe air bulb, produces a continuous flow of air—immediately, easily and comfortably offers relief and reduces the moisture and heat which contributes to the formation of the yeast infection, as well as reducing further risks of future bacterial and vaginal yeast infections. Finally, there is also a need for the vaginal drying air pump device and method for reducing excessive wetness, moisture and heat within the vagina with a constant and consistent flow of air; produced by manually squeezing a hand-operated easy-grip rubber pipet/syringe air bulb that can be operated by either the left or right hand and is portable enough to be easily carried in a woman's small purse, and ready for use whenever she needs drying.

U.S. Ser. No. 11/386,239 and US20070225781A1 issued to Vahid Saadat on Mar. 21, 2006 discloses an apparatus and methods for cooling and/or heating selected regions within a body are described herein, where an implantable system is used to cool or heat nerve bodies down to about 15° C. to diminish nerve impulses. In one embodiment, the system can include an implantable unit containing a pumping mechanism and/or various control electronics. The system has a cooling element. The cooling element can be a Peltier junction or a catheter through which hot or cold fluid flows. The heated portion of the Peltier junction can be cooled by a liquid heat transfer medium which absorbs the heat from the junction and dissipates the heat elsewhere.

Patent No. US20070021809A1 issued to Pamela Cole on Jul. 22, 2005 discloses a device and therapeutic method that provides topical cooling or warming to a user's vagina or vulva as a therapeutic benefit to and relief from maladies of these areas, where the device includes a protrusion that is placed into the user's vagina and a base that contacts the female genitals around the vagina. The device is cooled or warmed prior to or during use. The method includes the use of the device to provide a therapeutic effect and relief from maladies of a user's vagina or vulva.

Patent No. US20140058326 A1 issued to Kimberly Jo Cull on Feb. 27, 2014 discloses a cooling device and method which inhibits the infectious growth of pathogens in a body cavity, where the device has a shell having an internal chamber containing a freezable or cooling filler that reduces the body cavity temperature. The device may be comprised of an optional coating compound (agent) covering at least a portion of an outer surface of the shell and an optional stainless steel sleeve that covers the shell or optional stainless steel particles that are impregnated into the shell material to enhance both thermal conductance and cooling efficacy.

However, other vaginal drying devices reducing the moisture and temperature within the vagina; the ‘vagina wand’ and ‘virgin stick's’ common vaginal drying design has a severe inconvenience and drawback of causing over-tightness hindering penis penetration and vaginal burning due to the chemicals and components they contain.

Although the vagina wand and virgin stick have achieved considerable fame and commercial success in reducing excessive moisture in the vagina, there still has been a continuing need for improvement.

While various methods and vaginal drying devices for female hygiene exist, none relate to the design, method and function of eliminating and/or reducing the moisture, as well as reducing the heat in the vagina that produces and contributes to bacterial and yeast infections and lowering (or reducing) the risks for further infections according to the present invention.

### BRIEF SUMMARY OF THE INVENTION

One factor that makes the present invention unique is the “objective” of the design and function of the vaginal drying air pump. Another factor that makes the present invention unique is the method used to help reduce excessive heat-related conditions within the vaginal environment. The vaginal drying air pump a cooling device and cooling & drying method for reducing excessive moisture and excessive heat within the vaginal area to reduce (or decrease) and help inhibit heat-related conditions that produce or further promote bacterial and vaginal yeast infections; consisting of two pieces: a barrel (outer tube) made from the same material as a tampon plastic applicator and an inner tube also made from the same material as a tampon plastic applicator. The outer tube (barrel) with contoured tip and “Stop” indicator for comfortable and easy insertion into the vagina with air holes evenly spaced around the barrel's surface area allows hand-pumped air to freely flow through and out of the barrel, where the air flow distributes evenly on all sides into and within the vagina from the barrel, when the attached sealed syringe air bulb is manually hand-pumped and squeezed in a constant continuous motion. The inner tube is connected to the barrel (outer tube) in a fixed position by screwing together one end of the barrel with one end of the inner tube at the threads, and has the opposite inner tube's end, whereby a permanent air-tight rubber seal or rubber gasket is secured and connects to an easy grip rubber pipet/syringe air bulb to allow manual hand-operation of continuous squeezing motions to produce a continuous and constant flow of air (after the barrel is inserted into the vagina similar to the insertion and ease of a tampon plastic applicator). This continuous flow of air in the vagina, produced by hand-squeezing the easy grip rubber pipet/syringe air bulb, dries the excessive moisture and removes the excess heat, both of which contributes greatly to vaginal bacterial and vaginal yeast infections. In preferred embodiments, the various elements of the vaginal drying air pump; the barrel is approximately the same length and diameter of a regular tampon plastic applicator and also easy to clean with just two pieces that unscrews apart and can be swished around in soapy water, and rinsed. The vaginal drying air pump is portable enough to be easily carried in a woman's small purse, and ready for whenever she needs drying. The Barrel is approximately the same length and diameter of a regular sized tampon plastic applicator. The completed Vaginal Drying Air Pump device when screwed together has a total measurement of 7 inches in length. Air bulb measures 2.5 inches in diameter. Outer tube (Barrel) measures 0.5 inches in diameter and 3 inches in length. The Inner tube measures 0.33 inches in diameter and 2 inches in length. The Vaginal Drying Air Pump and Method, Process/System For Cooling & Drying Vagina: is the only device and/or apparatus which reduces the excessive moisture and reduces excessive heat within the vagina manually by squeezing a hand-operated (non-chemical, non-electrical, non-electronic, non-battery-operated or non-charged components) syringe air bulb unit that is safe to use, clean, portable and ready for use whenever needed.

### BRIEF DESCRIPTION OF THE DRAWINGS

A clear understanding of the key features of the invention summarized above may be had by reference to the appended drawings, which illustrates the design, function and method of the invention, although it will be understood that such drawings depict preferred embodiments of the invention and, therefore, are not to be considered as limiting its scope with regard to other embodiments which the invention is capable of contemplating. Accordingly:

FIG. 1 and FIG. 2 are illustrations of the design with its components and/or elements and function of the invention showing a simplified end-view diagram of a vaginal drying air pump; a female vaginal hygiene device or apparatus; a tampon plastic applicator that has been re-designed, newly modified and re-created with a new purpose, new objective, new design and new method—to function as a manually hand-operated squeezable air pump for removing and/or reducing excessive moisture and excessive heat from within the vagina, thereby reducing and minimizing the heated conditions within the vaginal environment that contribute to producing bacterial and vaginal yeast infections.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It is also understood that the term “whereby” is used herein to introduce a function or result at the end of a claim, and the term “wherein” is used herein in a dependent claim to recite an element or part more specifically. It will be further understood that the terms “comprises” and/or “comprising,” when used in this specification, specify the presence of stated features, steps, operations, elements, components and/or methods, but do not preclude the presence or addition of one or more other features, steps, operations, elements, methods, components, and/or groups thereof.

Unless otherwise defined, all terms (including technical, scientific, trade or industry terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques, steps, methods and features are disclosed. Each of these have individual benefits and each can also be used in conjunction with one or more, or in some cases all, of the other disclosed techniques, methods and/or features. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Newly re-designed, newly modified and re-created tampon plastic applicator design, functions and methods are discussed herein. In the following description, for purposes of explanation, numerous specific details are set forth in order to provide a thorough understanding of the present invention and method. It will be evident, however, to one skilled in the art that the present invention and method may be practiced without these specific details.

The present disclosure is to be considered as an exemplification of the invention and method, and is not intended to limit the invention and method to the specific embodiments illustrated by the figures or description below.

The present invention and method will now be described by referencing the appended figures representing preferred embodiments.

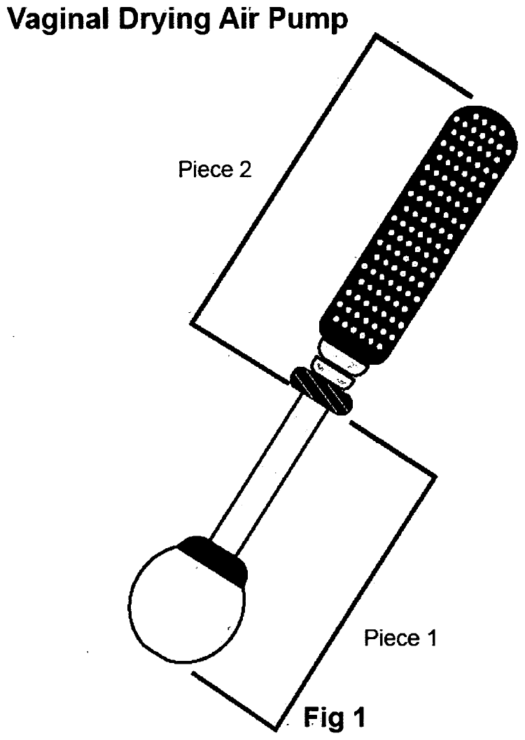

FIG. 1 depicts a 2D flat view of the completely assembled (screwed together) 2-piece components that may comprise a vaginal drying air pump with a re-designed tampon plastic applicator's outer tube (barrel) and inner tube indicating the contrast in color between the various sectional parts.

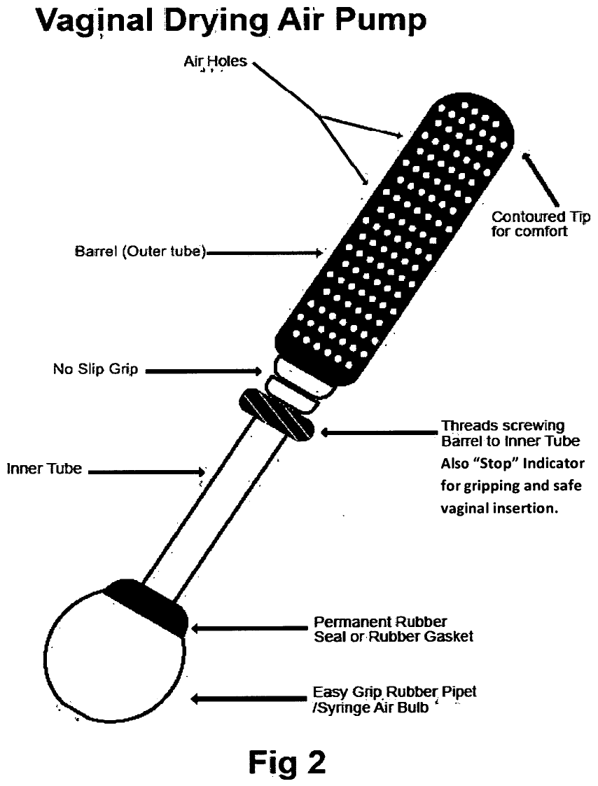

FIG. 2 depicts a 2D flat view drawing of the vaginal drying air pump's barrel (outer tube) view, top contoured tip for vaginal insertion comfort, air holes for a continuous evenly distributed flow of pumped air around barrel's surface via manually squeezing syringe air bulb, no-slip grip for secure finger or hand gripping for easily holding of device, thread connector screwing barrel to connect to inner tube with “Stop” indicator for safe vaginal insertion, inner tube for pathway/passage flow of hand-operated pumped air which is directed into barrel (outer tube), permanent rubber seal (rubber gasket) to secure and prevent pumped air from leaking out of syringe air bulb, and an easy-grip rubber pipet/syringe air bulb for manually hand-operated, easy squeezing to produce a constant flow of air into inner tube, thereby also flowing into barrel, being evenly distributed throughout barrel's air holes into the vagina to reduce moisture and heat.

While the present invention and method has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the design, function and method illustrated herein and of their operation can be made by those skilled in the art without departing from the spirit of this invention.

What is claimed is:

1) A vaginal drying air pump comprising: a) a re-designing of a tampon plastic applicator barrel (outer tube) with a modifying new function b) a re-designing of a tampon plastic applicator inner tube with a modifying new function c) a syringe air bulb with a permanent rubber sealing and rubber gasket d) easy gripping rubber pipet/syringe air bulb attaching to opposite end of inner tube and e) a newly created cooling and drying function with method, process and system for reducing excessive moisture and reducing excessive heat within the vaginal area; decreasing and inhibiting heat-related conditions producing and promoting bacterial and vaginal yeast infections; constructing a manual squeezable, hand-operated easy gripping rubber pipet/syringe air bulb connecting to an inner tube with the inner tube's opposite end screwing together at the connecting threads to an outer tube (barrel), distributing air flowing into barrel's air holes—by squeezing air into and within the vagina area.

2) A vaginal drying air pump of claim 1, further comprising: contouring barrel tip for comfort and easy vaginal inserting.

3) A vaginal drying air pump of claim 2, wherein said contouring barrel has air holes allowing evenly distributing of air flowing all around barrel's surface.

4) A vaginal drying air pump of claim 3, wherein said contouring barrel has a no-slip grip and ‘Stop’ allowing for easy gripping and holding of device or apparatus.

5) A vaginal drying air pump of claim 4, wherein said contouring barrel has threading lip screwing together and connecting barrel and inner tube as one piece.

6) A vaginal drying air pump comprising: an inner tube allowing a pathway for continuing and constant flowing of air into the barrel (outer tube).

7) A vaginal drying air pump of claim 6, further comprising: an easy gripping rubber pipet/syringe air bulb allowing for manual hand-operating, continuing easy squeezing and producing constant flowing of air into the barrel (outer tube) and throughout the air holes.

8) A vaginal drying air pump of claim 7, wherein said inner tube has a permanent rubber sealing and rubber gasket connecting the opening of the inner tube to the syringe air bulb, securing and preventing the flowing air from the squeezing of the syringe air bulb into the inner tube from leaking out.

9) A method, process and system of a manual hand-operating squeezable air flowing conduit, redesigned, recreated and newly modified from a plastic tampon applicator (barrel and inner tube), with a new function, new purpose and new objective; temporarily reducing excessive moisture and reducing excessive heat within the vagina, of claim 8, wherein comprising: a) providing a redesigning and a recreating of a plastic tampon applicator's outer barrel with evenly distributed air holes throughout the barrel's surface for air easily flowing into the vagina b) providing a redesigning and a recreating of a plastic tampon applicator's inner tube for air flowing into the outer barrel from manually hand squeezing the air bulb syringe sealed onto the inner tube's opposite end c) redesigning a tampon device or apparatus as a conduit for producing air flowing from an easy gripping rubber pipet/syringe air bulb's end portion, whereby the opening inner tube end easily screws together at the threads to the ‘Stop’ indicator for holding and safe vaginal inserting d) inserting the contouring tip of the assembled device within the vagina, as a tampon would be inserted e) squeezing the pipet/syringe air bulb in a continuing motion producing flowing air f) delivering a continuous flow of air through the conduit (or inner tube and barrel) g) allowing air flowing into and throughout air holes into the vagina area reducing excessive heat and excessive moisture.
